(19) United States
(12) Patent Application Publication
(10) Pub. No.: US 2019/0083930 A1
(43) Pub. Date: Mar. 21, 2019

(54) ENCLOSED SPACE INCLUDING A PHOTOCATALYTIC COATING AND A LIGHTING SYSTEM

(71) Applicant: AM TECHNOLOGY LIMITED, London (GB)

(72) Inventors: Massimo BERNARDONI, London (GB); Antonio CIANCI, London (GB)

(21) Appl. No.: 15/760,676

(22) PCT Filed: Sep. 16, 2016
(86) PCT No.: PCT/GB2016/052889

§ 371 Date: Mar. 16, 2018

(30) Foreign Application Priority Data
Sep. 16, 2015 (GB) 1516363.7

(51) Int. Cl. B01D 53/88; A61L 2/08; A61L 2/10; A61L 9/20; B01J 21/06; B01J 35/00; C09D 5/14

(52) U.S. Cl. CPC B01D 53/885; A61L 2/084; A61L 2/088; A61L 2/10; A61L 9/205; B01J 21/063; B01D 2257/91; C09D 5/14; B01D 2255/20707; B01D 2255/802; B01D 2257/302; B01D 2257/404; B01D 2257/708; B01J 35/004

(57) ABSTRACT

There is disclosed an enclosed space system through which vehicles may pass, the enclosed space system including interior surfaces, a photocatalytic coating on an interior surface of the enclosed space system, and a lighting system in attachment with an interior surface of the enclosed space system, the lighting system including light sources emitting wavelengths in the range between 340 nm and 450 nm, the lighting system arranged to illuminate the photocatalytic coating with the wavelengths in the range between 340 nm and 450 nm, wherein the photocatalytic coating is activatable by the wavelengths in the range between 340 nm and 450 nm. There is further disclosed use of light emitting diodes emitting wavelengths in the range between 340 nm and 450 nm to illuminate a surface coated with a photocatalytic coating, wherein the photocatalytic coating is activatable by the wavelengths in the range between 340 nm and 450 nm.

### BACKGROUND OF THE INVENTION

### 1. Field of the Invention

The field of the invention relates to enclosed spaces through which vehicles or pedestrians may pass, the enclosed spaces arranged or configured to reduce polluting gases. Such enclosed spaces may include transportation tunnels or car parks, for example.

### 2. Technical Background

NOx is a generic term for the mono-nitrogen oxides NO and NO2 (nitric oxide and nitrogen dioxide). They are produced from the reaction of nitrogen and oxygen gases in the air during combustion, especially at high temperatures. The two major emission sources are transportation vehicles and stationary combustion sources such as electric utilities and industrial boilers. A smaller amount, typically 5% of the total, is emitted as primary nitrogen dioxide, while the major proportion of atmospheric nitrogen dioxide is a secondary product of atmospheric chemistry (as a reaction with ozone). NOx is a potent greenhouse gas which also contributes to ground-level smog, ozone formation and acid rain. NOx emissions also contribute to the formation of fine particles and ozone smog that cost society increasing amounts of money from illnesses and deaths.

Unfortunately, air emissions targets remain largely unmet for key pollutants, including NOx, particulate matter (PM) and other volatile organic compounds (VOCs) that affect our environment and health. There is a need for investment in more novel and active physical and chemical solutions.

Scientific studies on photocatalysis started about four decades ago, and titanium dioxide has emerged as an excellent photocatalyst material for environmental purification.

TiO₂ has received a great deal of attention due to its chemical stability, non-toxicity, low cost, and other advantageous properties. TiO₂ is used in catalytic reactions acting as a promoter, a carrier for metals and metal oxides, an additive, or as a catalyst. Reactions carried out with TiO₂ catalysts, which can be powered by light (photocatalysis), include selective degradation of various chemicals such as SOx, NOx and VOCs.

Enclosed spaces through which vehicles or pedestrians may pass, such as tunnels or car parks inside buildings, are places in which polluting gases such as NOx may be produced or may already be present, and such polluting gases may harm human beings present in such spaces. There is a need to reduce the concentration of polluting gases such as NOx in enclosed spaces through which vehicles or pedestrians may pass. It is desirable to reduce in an energy efficient way the concentration of polluting gases such as NOx in enclosed spaces through which vehicles or pedestrians may pass.

### 3. Discussion of Related Art

JPH11324584A entitled “Tunnel Interior Finish Material made of Inorganic Sheet and Manufacture thereof”, has an English Abstract which discloses activation of a photocatalyst by lighting to decompose injurious ingredient in exhaust gas or organic substance stuck to interior finish material and easily cleaning the interior finish material by using an inorganic substance sheet fixed with photocatalyst particles on the surface as the tunnel interior finish material. The JPH11324584A English Abstract further discloses an aggregate and water and added to hydraulic coagulant containing 30-45 pts.wt. of silica containing at least 20 weight % of ultra fine particle silica of diameter under 1 μm, they are stirred in vacuum, poured in a form to form an inorganic sheet by cold setting, and it is used as tunnel inner facing material. The form is previously spread with pigment containing photocatalyst such as titanium oxide powder, and buried with an inorganic net for reinforce. Hereby, the photocatalyst particle on the surface of the inorganic sheet are activated by lighting in the tunnel or irradiation of the headlight of an automobile, NOx or SOx in exhaust gas is decomposed, and organic substance stuck to the sheet surface can be decomposed to facilitate cleaning.

### SUMMARY OF THE INVENTION

According to a first aspect of the invention, there is provided an enclosed space system through which vehicles may pass, the enclosed space system including interior surfaces, a photocatalytic coating on an interior surface of the enclosed space system, and a lighting system in attachment with an interior surface of the enclosed space system, the lighting system including light sources emitting wavelengths in the range between 340 nm and 450 nm, the lighting system arranged to illuminate the photocatalytic coating with the wavelengths in the range between 340 nm and 450 nm, wherein the photocatalytic coating is activatable by the wavelengths in the range between 340 nm and 450 nm. An advantage is that the light with the wavelengths in the range between 340 nm and 450 nm is effective in activating the photocatalytic coating, to reduce the presence of various harmful polluting gases in the enclosed space, and/or to eliminate bacteria and mould from the surface.

The enclosed space system may be one wherein the lighting system does not emit light with wavelengths below 340 nm. An advantage is that harmful effects to humans of light with wavelengths below 340 nm is avoided.

The enclosed space system may be a tunnel. An advantage is that the light with wavelengths in the range between 340 nm and 450 nm is effective in activating the photocatalytic coating, to reduce the presence of harmful polluting gases in the tunnel, and/or to eliminate bacteria and mould from the surface.

The enclosed space system may be a road tunnel. An advantage is that the light with wavelengths in the range between 340 nm and 450 nm is effective in activating the photocatalytic coating, to reduce the presence of harmful polluting gases from vehicle emissions in the tunnel, and/or to eliminate bacteria and mould from the surface.

The enclosed space system may be a rail tunnel. An advantage is that the light with wavelengths in the range between 340 nm and 450 nm is effective in activating the photocatalytic coating, to reduce the presence of harmful polluting gases from vehicle emissions, or from electric discharges, in the tunnel, and/or to eliminate bacteria and mould from the surface.

The enclosed space system may be a pedestrian tunnel. An advantage is that the light with wavelengths in the range between 340 nm and 450 nm is effective in activating the photocatalytic coating, to reduce the presence of harmful polluting gases in the tunnel, and/or to eliminate bacteria and mould from the surface.

The enclosed space system may be a car park in a building, in a ferry, or in a car train. An advantage is that the light with wavelengths in the range between 340 nm and 450 nm is effective in activating the photocatalytic coating, to reduce the presence of harmful polluting gases in the car park, and/or to eliminate bacteria and mould from the surface.

The enclosed space system may be one wherein the light sources emit wavelengths in the range between 340 nm and 389 nm.

The enclosed space system may be one wherein the light sources emit only light in the wavelength range from 340 nm to 450 nm. An advantage is improved energy efficiency of activating the photocatalytic coating, which may be a cement-based hydraulic binding photocatalytic coating. A further advantage is very little or no health risk to humans in the enclosed space.

The enclosed space system may be one wherein the light sources emit only light in the wavelength range from 340 nm to 389 nm. An advantage is improved efficiency of activating the photocatalytic coating. A further advantage is very lithe or no health risk to humans in the enclosed space.

The enclosed space system may be one wherein the light sources emit light in the wavelength range 340 nm to 389 nm and in the wavelength range 390 nm to 450 nm.

The enclosed space system may be one wherein the light emitted by the light sources in the wavelength range 340 nm to 450 nm is within a relatively narrow spectral distribution of radiation wavelengths. Examples of light sources with a relatively narrow spectral distribution of radiation wavelengths are light emitting diodes and lasers. An advantage is very lithe or no health risk to humans in the enclosed space.

The enclosed space system may be one wherein a surface coated with the photocatalytic coating is illuminated with the light in the wavelength range 340 nm to 450 nm at an intensity of light in the wavelength range 340 nm to 450 nm greater than 1.0 W/m².

The enclosed space system may be one wherein a surface coated with the photocatalytic coating is illuminated with the wavelengths in the range between 340 nm and 450 nm at an intensity in the range of 1.0 W/m² to 50 W/m².

The enclosed space system may be one wherein a surface coated with the photocatalytic coating is illuminated with the wavelengths in the range between 340 nm and 450 nm at an intensity in the range of 1.0 W/m² to 20 W/m².

The enclosed space system may be one wherein a surface coated with the photocatalytic coating is illuminated with the wavelengths in the range between 340 nm and 450 nm at an intensity in the range of 1.0 W/m² to 10 W/m².

The enclosed space system may be one wherein the light sources are one or more of fluorescent lamps, short wave lamps, gas discharge lamps, metal halide lamps and lasers.

The enclosed space system may be one wherein the light sources are light emitting diodes (LEDs). Advantages of LEDs include high power conversion efficiency and compact size.

The enclosed space system may be one wherein the light emitting diodes are arranged on a bar, or on a plurality of bars. An advantage is facilitation of maintenance.

The enclosed space system may be one wherein the light emitting diodes are arranged in the form of a frame including a plurality of LED spotlights, or in the form of a plurality of frames including a plurality of LED spotlights.

The enclosed space system may be one wherein use of the light emitting diodes emitting wavelengths in the range of 340 nm to 450 nm as light sources is scalable over a wide range of enclosed space sizes. An advantage is reusability or scalability of designs for different systems.

The enclosed space system may be one including a side wall coated with the photocatalytic coating, wherein the light sources emitting wavelengths in the range of 340 nm to 450 nm are directed towards the side wall.

The enclosed space system may be one including a ceiling coated with the photocatalytic coating, wherein the light sources emitting wavelengths in the range of 340 nm to 450 nm are directed towards the ceiling.

The enclosed space system may be one including a floor coated with the photocatalytic coating, wherein the light sources emitting wavelengths in the range of 340 nm to 450 nm are directed towards the floor.

The enclosed space system may be one wherein the system is arranged to reduce an amount of NOx gases in the enclosed space.

The enclosed space system may be one wherein the system is arranged to reduce an amount of SOx gases in the enclosed space.

The enclosed space system may be one wherein the system is arranged to reduce an occurrence of bacteria and molds on the surface.

The enclosed space system may be one wherein the system is arranged to reduce an amount of volatile organic compounds gases in the enclosed space.

The enclosed space system may be one wherein the photocatalytic coating is a cement-based hydraulic binding photocatalytic coating. An advantage is that the light with the wavelengths in the range between 340 nm and 450 nm is effective in activating the cement-based hydraulic binding photocatalytic coating, to reduce the presence of harmful polluting gases in the enclosed space, and/or to eliminate bacteria and mould from the surface.

The enclosed space system may be one wherein the photocatalytic coating is derived from a cement-based photocatalytic composition, which comprises:

(a) at least one cement binder;

(b) at least one photocatalyst;

(c) at least one cellulose ether;

(d) at least one fluidizing agent;

(e) at least one first calcareous filler in the form of particles of which at least 95% by weight has a dimension not greater than 100 μm;

(f) at least one second calcareous filler in the form of particles of which at least 95% by weight has a dimension not greater than 30 μm;

(g) at least one silane supported on an inorganic support in the form of powder.

The enclosed space system may be one wherein the photocatalytic composition comprises:

(a) from 15 to 60% by weight, preferably from 20 to 50% by weight, of at least one cement binder;

(b) from 0.5 to 12% by weight, preferably from 1 to 8% by weight, of at least one photocatalyst;

(c) from 0.02 to 3% by weight, preferably from 0.05 to 1.5% by weight, of at least one cellulose ether;

(d) from 0.05 to 5% by weight, preferably from 0.1 to 2% by weight, of at least one fluidizing agent;

(e) from 10 to 50% by weight, preferably from 15 to 35% by weight, of at least one first calcareous filler in the form of particles of which at least 95% by weight has a dimension not greater than 100 μm;

(f) from 10 to 50% by weight, preferably from 15 to 35% by weight, of at least one second calcareous filler in the form of particles of which at least 95% by weight has a dimension not greater than 30 μm;

(g) from 0.05 to 5% by weight, preferably from 0.01 to 3% by weight, of at least one silane supported on an inorganic support in the form of powder.

The enclosed space system may be one wherein the cement binder (a) is a Portland cement.

The enclosed space system may be one wherein the photocatalyst (eg. (b)) is photocatalytic titanium dioxide, mainly in anatase crystalline form.

The enclosed space system may be one wherein the photocatalytic titanium dioxide has a granulometry such as at least 95% by weight has a dimension not higher than 50 nm, preferably not higher than 20 nm.

The enclosed space system may be one wherein the photocatalytic titanium dioxide is in admixture with a non-photocatalytic titanium dioxide.

The enclosed space system may be one wherein the cellulose ether (c) has a Brookfield viscosity RVT at 20° C. from 100 to 70,000 mPa˜s, preferably from 100 to 30,000 mPa˜s, more preferably from 200 to 10,000 mPa˜s.

The enclosed space system may be one wherein the first calcareous filler (e) is in the form of particles of which at least 95% by weight has a dimension not greater than 70 μm, while the second calcareous filler (f) is in the form of particles of which at least 95% by weight has a dimension not greater than 20 μm.

The enclosed space system may be one wherein the first calcareous filler (e) is in the form of particles of which not more than 5% by weight has a dimension not greater than 30 μm, preferably not greater than 20 μm.

The enclosed space system may be one wherein the calcareous fillers (e) and (f) are present in a weight ratio (e)/(f) from 0.2 to 2.0, preferably from 0.5 to 1.5.

The enclosed space system may be one wherein the supported silane (g) is in the form of particles of which at least 95% by weight has a dimension not greater than 100μ, preferably not greater than 80μ.

The enclosed space system may be one further comprising: (h) at least one hydrophobized vinyl polymer, preferably a terpolymer of vinylchloride, ethylene and a vinyl ester CH₂═CH—O—C(═O)—R, wherein R is an alkyl, linear or branched, C₄-C₂₄.

The enclosed space system may be one further comprising: (i) at least one salt of a long chain carboxylic acid.

The enclosed space system may be one wherein water is added to the photocatalytic composition in a predetermined proportion, by mixing until a homogeneous and fluid product is obtained, and that product is applied to the interior surface of the enclosed space as the photocatalytic coating.

The enclosed space system may be one wherein the weight ratio between water and cement binder (a) is from 0.2 to 0.8.

The enclosed space system may be one wherein, after application and drying, the photocatalytic composition forms a coating layer having a thickness from 0.05 mm to 1 mm, preferably from 0.1 to 0.5 mm.

According to a second aspect of the invention, there is provided use in a tunnel of light emitting diodes emitting wavelengths in the range between 340 nm and 450 nm to illuminate an interior surface of the tunnel coated with a photocatalytic coating, wherein the photocatalytic coating is activatable by wavelengths in the range between 340 nm and 450 nm, and wherein the light emitting diodes do not emit light with wavelengths below 340 nm. Advantages include compact sources, which are energetically efficient, and which provide activation of the photocatalytic coating. An advantage is that harmful effects to humans of light with wavelengths below 340 nm is avoided.

The use may be one wherein the light emitting diodes emit wavelengths in the range between 340 nm and 389 nm.

The use may be one wherein the light emitting diodes emit only wavelengths in the range between 340 nm and 389 nm. An advantage is even more energetically efficient activation of the photocatalytic coating. An advantage is little or no health risks to humans.

The use may be one wherein the light emitting diodes emit wavelengths in the range between 340 nm and 389 nm and wavelengths in the range between 390 nm and 450 nm.

The use may be one wherein the surface coated with a photocatalytic coating is illuminated with the light emitting diodes at wavelengths in the range between 340 nm and 450 nm at an intensity greater than 1.0 W/m². An advantage is a high rate of reduction of harmful gases.

The use may be one wherein the surface coated with a photocatalytic coating is illuminated with the light emitting diodes at wavelengths in the range between 340 nm and 450 nm at an intensity in the range of 1.0 W/m² to 50 W/m².

The use may be one wherein the surface coated with a photocatalytic coating is illuminated with the light emitting diodes at wavelengths in the range between 340 nm and 450 nm at an intensity in the range of 1.0 W/m² to 20 W/m².

The use may be one wherein the surface coated with a photocatalytic coating is illuminated with the light emitting diodes at wavelengths in the range between 340 nm and 450 nm at an intensity in the range of 1.0 W/m² to 10 W/m².

There is provided use of light emitting diodes emitting wavelengths in the range between 340 nm and 450 nm to illuminate a surface coated with a photocatalytic coating, wherein the photocatalytic coating is activatable by the wavelengths in the range between 340 nm and 450 nm.

### BRIEF DESCRIPTION OF THE FIGURES

Aspects of the invention will now be described, by way of example(s), with reference to the following Figures, in which:

FIG. 1 shows a cross section of a tunnel including an illumination unit which emits light in a wavelength range between 340 nm and 450 nm. The dimensions of the tunnel are indicated, in cm.

FIG. 2 shows a cross section of a tunnel including an illumination unit which emits light in a wavelength range between 340 nm and 450 nm. The dimensions of the tunnel are not indicated.

### DETAILED DESCRIPTION

Enclosed spaces through which vehicles or pedestrians may pass, such as tunnels, car parks inside buildings, or car ferry interiors, are places in which polluting gases such as NOx may be produced or may already be present such as by traveling in to the enclosed space from outside, and such polluting gases may harm human beings present in such enclosed spaces. Tunnels may be road vehicle tunnels, such as the Blackwall Tunnel in London, United Kingdom, under the river Thames. In road vehicle tunnels, polluting gases such as NOx may be produced by internal combustion engines. Tunnels may be rail tunnels, such as the Waterloo & City line tunnels in London, under the river Thames, or the Belsize tunnels in London on the Midland main line north of St Pancras station. In rail tunnels, polluting gases such as NOx may be produced by internal combustion engines, or by electric discharge. In car parks inside buildings, or in car ferry interiors, polluting gases such as NOx may be produced by internal combustion engines.

Examples of vehicles include automobiles, trucks, buses, trains, motorbikes, and bicycles.

Light emitting sources may be arranged to illuminate a photocatalytic coating in an enclosed space eg. in a tunnel. Light sources may emit light in the wavelength range 340 nm to 450 nm. Light sources may emit essentially only wavelengths in the range 340 nm to 389 nm, or light sources may emit light in the wavelength range 340 nm to 389 nm and in the wavelength range 390 nm to 450 nm. Known light sources which may emit light in the wavelength range 340 nm to 450 nm include fluorescent lamps, short wave lamps, gas discharge lamps, metal halide lamps, light emitting diodes and lasers. The known light sources which may emit light in the wavelength range 340 nm to 450 nm which include fluorescent lamps, short wave lamps, gas discharge lamps, metal halide lamps, light emitting diodes and lasers, may be powered by mains electricity. The photocatalytic coating may include TiO₂. The photocatalytic coating may provide selective degradation of various chemicals such as SOx, NOx and VOCs.

Light emitting diode (LED) sources may be arranged to illuminate a photocatalytic coating in an enclosed space, eg. in a tunnel. LED sources may be in the form of a bar of LED sources. LED sources may be in the form of a frame including a plurality of LED spotlights. A bar of LED sources typically has the advantages of reduced assembly cost, and reduced installation cost, when compared to a frame including a plurality of LED spotlights.

LED light sources include LED light sources emitting wavelengths in the range 340 nm to 389 nm. An example is a Nichia U365 LED (supplied by Nichia Corporation, Tokushima 774-8601, Japan) which has a peak wavelength at 365 nm, a spectrum half width of 9 nm and a radiant flux of 780 mW. A further example is a Nichia U385 LED (supplied by Nichia Corporation, Tokushima 774-8601, Japan) with a peak wavelength at 385 nm, a spectrum half width of 10 nm, and a radiant flux of 900 mW. For both the U365 and U385 LEDs, the intensity falls to half its value along the normal axis at about 65 degrees to the normal axis, and the intensity falls to a quarter its value along the normal axis at about 80 degrees to the normal axis. An advantage of LED light sources is precise control of a relatively narrow spectral distribution of radiation wavelengths, in contrast to for example light sources which emit light over a broad range of wavelengths.

The Nichia U365 LED is an example of an LED which essentially emits only light in the wavelength range 340 nm to 389 nm; the Nichia U385 LED is an example of an LED which emits light in the wavelength range 340 nm to 389 nm and light in the wavelength range 390 nm to 450 nm.

An irradiation intensity for light in the wavelength range 340 nm to 450 nm for inducing photo catalytic activity in a photo catalytic coating (eg. a paint) (such as one described herein) suitable for removing NOx at a sufficient rate, or for having anti-bacterial properties, has been found in experiments to be 1.0 W/m², or more. An irradiation intensity for light in the wavelength range 340 nm to 450 nm for inducing photo catalytic activity in a photo catalytic coating (eg. a paint) (such as one described herein) suitable for removing NOx at a very high rate, or for having anti-bacterial properties, has been found in experiments to be 20 W/m², or more. An irradiation intensity for light in the wavelength range 340 nm to 450 nm for inducing photo catalytic activity in a photo catalytic coating (eg. a paint) (such as one described herein) suitable for removing NOx at a sufficient rate, or for having anti-bacterial properties, may be in the range of 1.0 W/m² to 10 W/m², or in the range of 1.0 W/m² to 20 W/m², or in the range of 1.0 W/m² to 50 W/m².

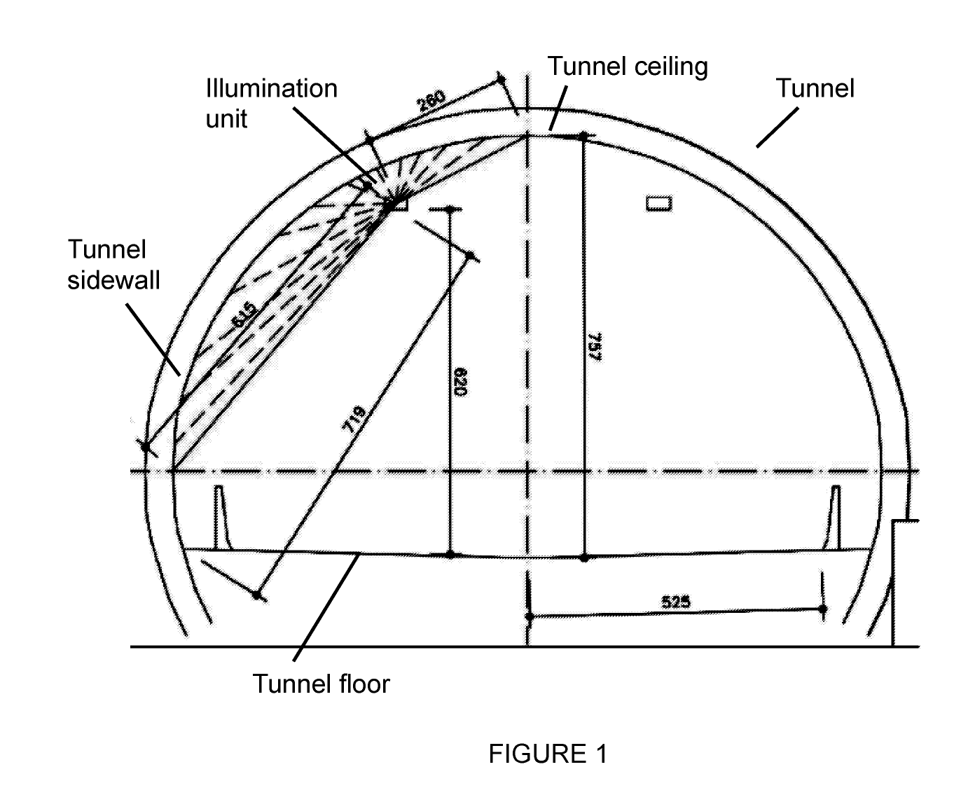

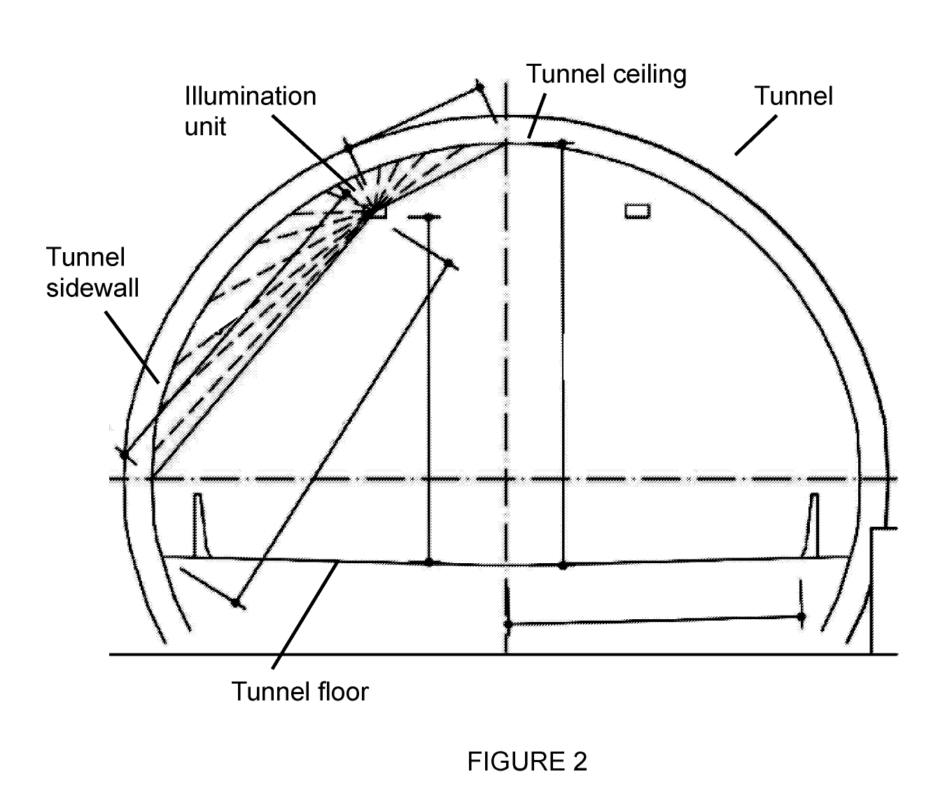

In an example, a lighting unit includes 3 bars for the lighting unit body; each bar provides 4 LEDs. Thus, each lighting unit consists of 12 LEDs. Each lighting unit body is able to meet the lighting requirements on average of about 1 square meter of surface to be illuminated/irradiated. Each lighting unit body radiates Wrad 9.95 (net of losses of lenses and protective glass) and consumes a.c. 25 WPot. Considering, therefore, a useful tunnel surface profile length of 14 m to be illuminated, our simulations show that, using Nichia U365 LEDs, 6 lighting unit bodies (3×2 tracks) are required per meter of tunnel length, when the lighting unit bodies are arranged near to the ceiling of the tunnel, to provide a high rate of NOx removal. An example tunnel cross section, showing a lighting unit light source disposed near to the tunnel ceiling, is shown in FIG. 1. In FIG. 1, distances are given in cm, but are provided by way of example only. An example tunnel cross section, showing a lighting unit light source disposed near to the tunnel ceiling, is shown in FIG. 2. In FIG. 2, distances are not given.

A simple model calculation supports the simulation results. Six lighting units produce about 60 W of net radiated output. This can provide an intensity of 1.0 W/m² over an area of 60 m². Modelling the 60 W of net radiated output to fall on a hemisphere of radius r, area 2πr², a hemispherical area of 60 m² corresponds to a radius r of about 3.1 m. Hence points on a tunnel wall or ceiling within 3.1 m of a lighting unit will meet the target intensity of 1.0 W/m² in this simple model. This simple model already guarantees sufficient illumination of a substantial portion of example tunnel surfaces: see e.g. FIG. 1, in which a tunnel surface profile length of about 14 m is illuminated.

Our calculations show that the use of LEDs is an energy efficient way of inducing photo catalytic activity in a photo catalytic coating (eg. a paint) inside a tunnel. In the above example, only 6*25 W=150 W of source mains power is required. It is surprising that devices as small as LEDs are suitable for inducing a high level of photo catalytic activity in a photo catalytic coating (eg. a paint) inside a tunnel in an energy efficient way. Typically, lighting in tunnels has used large, bulky light emitting units.

Because our simulation shows that 6 lighting unit bodies, each including 12 LEDs, hence 72 LEDs in total, are suitable to provide a high rate of NOx removal per metre length of tunnel (in which a tunnel surface profile length of 14 m is illuminated), the use of LEDs emitting wavelengths in the range of 340 nm to 450 nm is scalable over a wide range of tunnel sizes, because the number of LEDs used per metre length of tunnel can be scaled up or down with increasing or with decreasing tunnel size, respectively.

In an example, light emitting sources emitting wavelengths in the range of 340 nm to 450 nm may be directed towards a ceiling of a tunnel coated with a photocatalytic coating. In an example, light emitting sources emitting wavelengths in the range of 340 nm to 450 nm may be directed towards side walls of a tunnel coated with a photocatalytic coating. In an example, light emitting sources emitting wavelengths in the range of 340 nm to 450 nm may be directed towards a ceiling and towards side walls of a tunnel coated with a photocatalytic coating. In an example, light emitting sources emitting wavelengths in the range of 340 nm to 450 nm may be directed towards a floor of a tunnel coated with a photocatalytic coating, for example in a rail tunnel.

Exposure of human beings to low wavelength radiation may be considered to provide some health risk. Excessive exposure to one kind of radiation (shorter-wave, germicidal) can damage tissue. It is present increasingly in sunlight with the thinning of the protective ozone layer, and in tanning salons and halogen lamps. Yet light with wavelengths in the range between 340 nm and 450 nm in natural daylight is required for both human physical and mental health, muscle strength, civilized behavior, energy and learning. There is no evidence of health risks for human health for exposure to light with wavelength in the range between 340 nm and 450 nm. Therefore, for spaces in which humans will be present, it is advantageous to illuminate photocatalytic coatings with light with wavelength in the range between 340 nm and 450 nm. In particular, for spaces in which humans will be present, it is advantageous to ensure that wavelengths below 340 nm are not used to illuminate photocatalytic coatings.

Example Photocatalytic Compositions

There are provided cement-based photocatalytic compositions, and use thereof for obtaining water paints, in particular for outdoor applications, or for applications in enclosed spaces.

There are provided cement-based hydraulic binding photocatalytic compositions, and use thereof for obtaining water paints, in particular for outdoor applications, or for applications in enclosed spaces.

Example cement-based photocatalytic compositions are provided, which comprise: (a) at least one cement binder; (b) at least one photocatalyst; (c) at least one cellulose ether; (d) at least one fluidizing agent; (e) at least one first calcareous filler in the form of particles of which at least 95% by weight has a size not greater than 100 μm; (f) at least one second calcareous filler in the form of particles of which at least 95% by weight has a size not greater than 30 μm; (g) at least one silane supported on an inorganic support in the form of powder. Such compositions can be employed as a water paint for obtaining wall coatings with very low thickness, in particular for outdoor applications, or for applications in enclosed spaces, which ensure a high and stable photocatalytic effect over time even with relatively low quantities of photocatalyst, generally lower than 10% by weight, with optimal results in terms of uniformity of the coating and resistance of the same to weathering agents.

Photocatalysis is a natural phenomenon that regards some substances, known as photocatalysts, which—when irradiated with light of suitable wavelength—are capable of catalyzing some chemical reactions. In particular, in the presence of air and light, oxidative processes are activated on a surface containing a photocatalytic substance that lead to the transformation and/or decomposition of organic and inorganic polluting substances (microbes, nitrogen oxides, polycondensate aromatic products, benzene, sulfur dioxide, carbon monoxide, formaldehyde, acetaldehyde, methanol, ethanol, benzene, ethylbenzene, methylbenzene, nitrogen monoxide and dioxide). Such polluting and/or toxic substances are transformed, through the photocatalysis process, into innocuous substances that can be washed away by rain water or via washing, such as sodium nitrate (NaNO₃), calcium sulfate (CaSO₄), calcium nitrate (Ca(NO₃)₂) and calcium carbonate (CaCO₃). Photocatalytic processes can then be used for considerably reducing the pollutants present in the environment, such as those produced by the exhaust gases of automobiles, factories, home heating and other sources, and at the same time eliminate dirt, mold, and bacteria that degrade the external surfaces of buildings or other structures.

The photocatalysts are generally metal compounds such as titanium dioxide, TiO₂, the most active and most used, zinc oxide, ZnO, and other oxides and sulfides (CeO₂, ZrO₂, SnO₂, CdS, ZnS, etc.).

Much effort has been expended to provide compositions containing a photocatalyst to be used for coating building surfaces, which can be applied with the techniques commonly employed in the building industry; such compositions ensure a significant and enduring photocatalytic effect, simultaneously ensuring a satisfactory aesthetic effect, as well as of course at non-excessive costs, so as to allow the application thereof on a large scale.

According to the prior art, the photocatalytic product is usually incorporated in formulations of paints or varnishes with substantially organic base of conventional type. Nevertheless, such formulations, given that they are of organic nature, undergo the action of transformation and/or decomposition catalyzed by the photocatalyst, so that the properties of the applied coating are degraded over time, with detachment and pulverization phenomena, as well as causing a quick decay of the original photocatalytic properties.

Also known in the art are cement-based compositions which comprise a photocatalyst. For example, in the patent application WO 2009/013337, photocatalytic compositions are described which comprise: a hydraulic binder; a polycarboxylic or acrylic superfluidizing agent; a cellulose ether with viscosity comprised between 10,000 and 120,000 mPa˜s; an adhesive agent; a calcareous, silicic or silicocalcareous filler; a photocatalyst. Such compositions would be provided with rheological properties such to render them particularly suitable for the application on large surfaces, without dripping or deformations.

In the patent application WO 2013/018059, a photocatalytic powder paint is described for use diluted in water, which comprises: Portland cement combined with photocatalytic titanium dioxide in nanoparticle form; a calcareous inert substance with maximum particle size lower than 100 μm; cellulose with viscosity lower than 1000 mPa˜s; a fluidizing agent; an anti-foaming agent; a vinyl polymer; pigments. Such composition also comprises at least one of the following additives: metakaolin, calcium formate and diatomaceous earth.

The Applicant has faced the technical problem of providing a cement-based photocatalytic composition, usable for obtaining water paints, namely wall coatings with very low thickness, in particular for outdoor applications, or for applications in enclosed spaces, which is capable of:

(a) ensuring a high photocatalytic effect that is stable over time, also with relative low quantities of photocatalyst, generally lower than 10% by weight;

(b) allowing the preparation and application of the water paint with conventional means, such as those used for common painting works, with optimal results in terms of uniformity of the coating and resistance of the same to weathering agents;

(c) using products devoid of toxic or dangerous effects, without using heavy metals and organic solvents, in particular aromatic solvents, so as to obtain a product with a content of volatile organic compounds (VOC) lower than 0.35 g/l.

These and further objects that will be better illustrated hereinbelow have been achieved by the Applicant by means of a cement-based photocatalytic composition as defined in the following description and enclosed clauses, which allows obtaining, in addition to the above-described results, also an improved reflectance of the visible radiation, due in particular to the use of a combination of calcareous fillers having different particle size.

In addition, the addition of a silane in powder form as better described hereinbelow ensures greater hydrophobicity to the water paint, and hence improved resistance to the action of weathering agents.

In a first aspect, the present disclosure therefore regards a cement-based photocatalytic composition, which comprises:

(a) at least one cement binder;

(b) at least one photocatalyst;

(c) at least one cellulose ether;

(d) at least one fluidizing agent;

(e) at least one first calcareous filler in the form of particles of which at least 95% by weight has a size not greater than 100 μm;

(f) at least one second calcareous filler in the form of particles of which at least 95% by weight has a size not greater than 30 μm;

(g) at least one silane supported on an inorganic support in the form of powder.

Preferably, the photocatalytic composition comprises:

(a) from 15 to 60% by weight, more preferably from 20 to 50% by weight, of at least one cement binder;

(b) from 0.5 to 12% by weight, more preferably from 1 to 8% by weight, of at least one photocatalyst;

(c) from 0.02 to 3% by weight, more preferably from 0.05 to 1.5% by weight, of at least one cellulose ether;

(d) from 0.05 to 5% by weight, more preferably from 0.1 to 2% by weight, of at least one fluidizing agent;

(e) from 10 to 50% by weight, more preferably from 15 to 35% by weight, of at least one first calcareous filler in the form of particles of which at least 95% by weight has a size not greater than 100 μm;

(f) from 10 to 50% by weight, more preferably from 15 to 35% by weight, of at least one second calcareous filler in the form of particles of which at least 95% by weight has a size not greater than 30 μm;

(g) from 0.05 to 5% by weight, more preferably from 0.01 to 3% by weight, of at least one silane supported on an inorganic support in the form of powder.

In the scope of the present description and of the enclosed clauses and Claims, the quantities of the various components of the photocatalytic composition are expressed, except where differently indicated, as percentages by weight with respect to the overall weight of the composition itself.

In a second aspect, the present disclosure regards the use of a cement-based photocatalytic composition as defined above for coating building structures in order to reduce the presence of polluting agents.

In addition, the present disclosure regards the use of a cement-based photocatalytic composition as defined above for coating surfaces made of metal, wood or plastic material, e.g. polyvinylchloride (PVC). With regard to the cement binder (a), this is generally made of a hydraulic cement material in powder form in dry state, which, when mixed with water, forms a plastic material that is capable of consolidating and hardening after a time sufficient to allow the application thereof in the plastic state. Preferably, the cement binder is Portland cement.

Preferably, the photocatalyst (b) is titanium dioxide in photocatalytic form, i.e. mainly in anatase crystalline form. The photocatalytic titanium dioxide preferably has a particle size such that at least 95% by weight has a size not greater than 50 nm, more preferably not greater than 20 nm. Preferably the photocatalytic titanium dioxide has a surface area comprised between 100 and 500 m²/g. The photocatalytic titanium dioxide can also be used in admixture with non-photocatalytic titanium dioxide, for example in rutile crystalline form, which allows imparting an intense white color to the composition.

Preferably, the non-photocatalytic titanium dioxide is present in a quantity from 0.5 to 20% by weight, more preferably from 1 to 15% by weight.

As regards the cellulose ether (c), this preferably has a Brookfield viscosity RVT at 20° C. from 100 to 70,000 mPa˜s, more preferably from 100 to 30,000 mPa˜s, even more preferably from 200 to 10,000 mPa˜s. The viscosity can be measured, for example, on a 2% solution by weight in water. In particular, the cellulose ether can be selected from: ethylcellulose, hydroxypropylcellulose, methylhydroxypropylcellulose, methylcellulose, carboxymethylcellulose, methylcarboxyethylcellulose, or mixtures thereof.

Products of this type can be found on the market, for example with the trademarks Culminal™, Walocel™ and Tylose™.

The fluidizing agent (d) can be selected from the products commonly employed in the cement field. These are usually vinyl or acrylic polymers, such as for example: polyvinylacetate, polyvinylversatate, polybutylacrylate or copolymers thereof (commercial products by Elotex). Preferably, the fluidizing agent is a superfluidizing agent, e.g. polycarboxylate, more specifically a copolymer from an unsaturated mono- or dicarboxylic acid and a polymerizable unsaturated comonomer. Examples of unsaturated mono- or dicarboxylic acids include: acrylic acid, methacrylic acid, crotonic acid, maleic acid, fumaric acid, itaconic acid, citraconic acid, and the like. Examples of polymerizable unsaturated comonomers include: polyalkylene glycol mono(meth)acrylate (e.g.: triethylene glycol monoacrylate and polyethylene glycol monoacrylate, in which the polyethylene glycol has an average molecular weight from 200 to 1000). Products of this type can be found on the market, for example with the trademark Melflux™.

With regard to the calcareous fillers (e) and (f), the first calcareous filler is in the form of particles of which at least 95% by weight has a size not greater than 100 μm, preferably not greater than 70 μm, while the second calcareous filler is in the form of particles of which at least 95% by weight has a size not greater than 30 μm, preferably not greater than 5 μm. Preferably, the first calcareous filler is in the form of particles of which not more than 5% by weight has a size not greater than 30 μm, preferably not greater than 20 μm. The calcareous fillers, defined for example in the UNI EN 12620:2008 standard, are finely subdivided calcareous minerals, mainly containing calcium carbonate (generally the calcium carbonate content is at least equal to 75% by weight).

Preferably, the calcareous fillers (e) and (f) are present in a weight ratio (e)/(f) between 0.2 and 2.0, more preferably between 0.5 and 1.5. The Applicant believes that the addition of the second calcareous filler, having finer particle size than the first, allows obtaining a coating of greater quality since the smaller granules fill the interstices present between the particles of the other materials, in particular between the particles of the photocatalyst.

With regard to the silane supported on an inorganic support in the form of powder (g), this is generally an organic silane supported on an inorganic support, such as silica or silicates. Preferably the supported silane is in the form of particles of which at least 95% by weight has a size not greater than 100μ, preferably not greater than 80μ.

Preferably, the silane is an alkyltrialkoxy silane of formula R₁Si(OR₂)₃, where R₁ is an alkyl C₁-C₁₈, preferably C₄-C₁₂, linear or branched, while the groups R₂, equal to or different from each other, are alkyls, linear or branched, C₁-C₆, preferably C₁-C₄. For example, the silane is i-butyltriethoxysilane, noctyltriethoxysilane, i-octyltriethoxysilane.

Preferably, the photocatalytic composition in accordance with the present disclosure further comprises at least one hydrophobized vinyl polymer (h), which allows further increasing the hydrophobic properties of the water paint. Such polymer (h), available in powder form, can be preferably added in a quantity from 1 to 20% by weight, more preferably from 3 to 10% by weight.

Preferably, the hydrophobized vinyl polymer is a vinylchloride, ethylene and vinyl ester terpolymer CH₂═CH—O—C(═O)—R, where R is an alkyl, linear or branched, C₄-C₂₄, e.g. vinyl laurate. Products of this type can be found on the market, for example with the trademark Vinnapas™.

Still as hydrophobizing agent, at least one salt of a long chain carboxylic acid (i) can be added to the photocatalytic compositions in accordance with the present disclosure, for example calcium stearate, and the like. The quantity of said salt is generally comprised between 0.01 and 5% by weight, more preferably between 0.1 and 2% by weight.

The photocatalytic composition in accordance with the present disclosure can also comprise further additives commonly used in this product type, such as: anti-foaming agents, pigments, aerating additives, metakaolin, calcium formate, diatomaceous earth, etc.

The photocatalytic composition in accordance with the present disclosure can be produced in accordance with known techniques, via mixing of the various components in dry state in any order, using a suitable mechanical mixer, e.g. a planetary mixer, for a time sufficient for obtaining good homogenization.

In order to prepare the water paint, water is added to the photocatalytic composition in the predetermined proportion, mixing until a homogeneous and fluid product is obtained.

The weight ratio between water and cement binder (a) can vary within wide limits as a function of the specificity of the used components and of the application technique that one wishes to employ. The water/binder weight ratio is generally comprised between 0.2 and 0.8.

The application of the water paint can be made with conventional means, such as those used for common painting works, like brushes and rollers, or even spatulas, trowels, airless pumps, etc. The application can occur on buildings of various type, such as wall structures, both external and internal, tiles, slabs, prefabricated structures, cement buildings such as sound absorbent barriers and new jersey barriers, tunnels, exposed concrete, constituting part of urban buildings or street furniture. After application and drying, the thickness of the photocatalytic composition layer can vary within wide limits as a function of the building and of the photocatalytic effect that one wishes to obtain.

Generally, a thickness from 0.05 mm to 1 mm, more preferably from 0.1 to 0.5 mm is sufficient.

The following examples are provided for merely exemplifying purposes of the present disclosure and must not be intended as limiting the protective scope defined by the enclosed clauses and Claims.

### EXAMPLE 1

A photocatalytic composition was obtained in accordance with the present disclosure by mixing the following components in the quantities reported in Table 1.

TABLE 1

| | | Quantity |
|---|---|---|
| | | (% by |
| Component | Commercial name | weight) |
| Portland cement | — | 40 |
| Photocatalytic | CristalActiv ™ PC500 | 5 |
| titanium dioxide | | |
| Cellulose ether | Culminal ™ MHPC 500 | 0.8 |
| (methylhydroxypropyl- | PF | |
| cellulose) | | |
| Superfluidizing agent | Melflux ™ 2651 F | 0.5 |
| Micronized calcareous | Lithos ™ Mineraria | 20 |
| filler | Lithocarb GR60 | |
| (≥95% with size ≤60 μm) | | |
| Calcareous | Imerys ™ #10 white | 20 |
| ultrafiller | | |
| (≥95% with size ≤20 μm) | | |
| Silane in powder form | Protectosil ™ 851 | 0.5 |
| Metakaolin | — | 2.2 |
| Non-photocatalytic | Tioxide R-XL | 5 |
| titanium dioxide | | |
| Hydrophobized vinyl | Vinnapas ™ 8034 H | 4 |
| polymer | | |
| Anti-foaming agent | Defomex ™ AP 199 | 1.5 |
| Calcium stearate | — | 0.5 |

A water paint was prepared by mixing the aforesaid composition with water in a 60% weight ratio. The water paint was applied on a sample with an average thickness equal to 0.3 mm, and solar light reflectance and heat emittance characteristics thereof were measured. The results are reported in Table 2.

TABLE 2

| Property | Standard | Measured value |
|---|---|---|
| Solar reflectance | ASTM E1980-11 | 112 |
| index (SRI) | | |
| Solar reflectance | ASTM C1549-09 | 88.8% |
| Thermal emittance | ASTM C1371-04a | 0.86 |

The solar reflectance is the fraction of the incident solar radiation that is reflected by an irradiated surface; it can vary from zero for a totally absorbent surface, to 1 (i.e. 100%), for a perfectly reflecting surface. The thermal emissivity is the ratio between the thermal radiation actually emitted by a surface and the maximum theoretical emission at the same temperature; this also varies from 0 to 1. A cover surface with high solar reflectance absorbs only a small part of the incident solar radiation. In addition, most of the solar energy that was absorbed is returned to the outside environment if the cover surface has equally high thermal emissivity.

The obtained product can thus be labelled “Energy Star”, ensuring a solar reflectance greater than 65%.

Clauses

1. Cement-based photocatalytic composition, which comprises:

(a) at least one cement binder;

(b) at least one photocatalyst;

(c) at least one cellulose ether;

(d) at least one fluidizing agent;

(e) at least one first calcareous filler in the form of particles of which at least 95% by weight has a dimension not greater than 100 μm;

(f) at least one second calcareous filler in the form of particles of which at least 95% by weight has a dimension not greater than 30 μm;

(g) at least one silane supported on an inorganic support in the form of powder.

2. Photocatalytic composition according to clause 1, which comprises:

(a) from 15 to 60% by weight, preferably from 20 to 50% by weight, of at least one cement binder;

(b) from 0.5 to 12% by weight, preferably from 1 to 8% by weight, of at least one photocatalyst;

(c) from 0.02 to 3% by weight, preferably from 0.05 to 1.5% by weight, of at least one cellulose ether;

(d) from 0.05 to 5% by weight, preferably from 0.1 to 2% by weight, of at least one fluidizing agent;

(e) from 10 to 50% by weight, preferably from 15 to 35% by weight, of at least one first calcareous filler in the form of particles of which at least 95% by weight has a dimension not greater than 100 μm;

(f) from 10 to 50% by weight, preferably from 15 to 35% by weight, of at least one second calcareous filler in the form of particles of which at least 95% by weight has a dimension not greater than 30 μm;

(g) from 0.05 to 5% by weight, preferably from 0.01 to 3% by weight, of at least one silane supported on an inorganic support in the form of powder.

3. Photocatalytic composition according to anyone of the preceding clauses, wherein the cement binder (a) is a Portland cement.

4. Photocatalytic composition according to anyone of the preceding clauses, wherein the photocatalyst (b) is photocatalytic titanium dioxide, mainly in anatase crystalline form.

5. Photocatalytic composition according to clause 4, wherein the photocatalytic titanium dioxide has a granulometry such as at least 95% by weight has a dimension not higher than 50 nm, preferably not higher than 20 nm.

6. Photocatalytic composition according to clause 4 or 5, wherein the photocatalytic titanium dioxide is in admixture with a non-photocatalytic titanium dioxide.

7. Photocatalytic composition according to anyone of the preceding clauses, wherein the cellulose ether (c) has a Brookfield viscosity RVT at 20° C. from 100 to 70,000 mPa˜s, preferably from 100 to 30,000 mPa˜s, more preferably from 200 to 10,000 mPa˜s.

8. Photocatalytic composition according to anyone of the preceding clauses, wherein the first calcareous filler (e) is in the form of particles of which at least 95% by weight has a dimension not greater than 70 μm, while the second calcareous filler

(f) is in the form of particles of which at least 95% by weight has a dimension not greater than 20 μm.

9. Photocatalytic composition according to anyone of the preceding clauses, wherein the first calcareous filler (e) is in the form of particles of which not more than 5% by weight has a dimension not greater than 30 μm, preferably not greater than 20 μm.

10. Photocatalytic composition according to anyone of the preceding clauses, wherein the calcareous fillers (e) and (f) are present in a weight ratio (e)/(f) from 0.2 to 2.0, preferably from 0.5 to 1.5.

11. Photocatalytic composition according to anyone of the preceding clauses, wherein the supported silane (g) is in the form of particles of which at least 95% by weight has a dimension not greater than 100μ, preferably not greater than 80μ.

12. Photocatalytic composition according to anyone of the preceding clauses, further comprising: (h) at least one hydrophobized vinyl polymer, preferably a terpolymer of vinylchloride, ethylene and a vinyl ester CH₂═CH—O—C(═O)—R, wherein R is an alkyl, linear or branched, C₄-C₂₄.

13. Photocatalytic composition according to anyone of the preceding clauses, further comprising: (i) at least one salt of a long chain carboxylic acid.

14. Use of a cement-based photocatalytic composition according to anyone of clauses from 1 to 13, for coating building artifacts in order to reduce the presence of polluting agents.

15. Use according to clause 14, wherein water is added to the photocatalytic composition in a predetermined proportion, by mixing until a homogeneous and fluid product is obtained.

16. Use according to clause 15, wherein the weight ratio between water and cement binder (a) is from 0.2 to 0.8.

17. Use according to anyone of clauses from 14 to 16, wherein, after application and drying, the photocatalytic composition forms a coating layer having a thickness from 0.05 mm to 1 mm, preferably from 0.1 to 0.5 mm.

18. Use of a cement-based photocatalytic composition according to any one of the clauses from 1 to 13, for coating surfaces made of metal, wood or plastic material, e.g. polyvinylchloride (PVC).

Note

It is to be understood that the above-referenced arrangements are only illustrative of the application for the principles of the present invention. Numerous modifications and alternative arrangements can be devised without departing from the spirit and scope of the present invention. While the present invention has been shown in the drawings and fully described above with particularity and detail in connection with what is presently deemed to be the most practical and preferred example(s) of the invention, it will be apparent to those of ordinary skill in the art that numerous modifications can be made without departing from the principles and concepts of the invention as set forth herein.

1. An enclosed space system through which vehicles may pass, the enclosed space system including interior surfaces, a photocatalytic coating on an interior surface of the enclosed space system, and a lighting system in attachment with an interior surface of the enclosed space system, the lighting system including light sources emitting wavelengths in the range between 340 nm and 450 nm, the lighting system arranged to illuminate the photocatalytic coating with the wavelengths in the range between 340 nm and 450 nm, wherein the photocatalytic coating is activatable by the wavelengths in the range between 340 nm and 450 nm.

2. The enclosed space system of claim 1, wherein the lighting system does not emit light with wavelengths below 340 nm.

3. The enclosed space system of claim 1, (i) wherein the enclosed space system is a tunnel, or (ii) wherein the enclosed space system is a road tunnel, or (iii) wherein the enclosed space system is a rail tunnel, or (iv) wherein the enclosed space system is a pedestrian tunnel.

4-6. (canceled)

7. The enclosed space system of claim 1, wherein the enclosed space system is a car park in a building, a car park in a ferry, or a car park in a car train.

8-12. (canceled)

13. The enclosed space system of claim 1, (i) wherein a surface coated with the photocatalytic coating is illuminated with the wavelengths in the range between 340 nm and 450 nm at an intensity greater than 1.0 W/m², or (ii) wherein a surface coated with the photocatalytic coating is illuminated with the wavelengths in the range between 340 nm and 450 nm at an intensity in the range of 1.0 W/m² to 50 W/m², or (iii) wherein a surface coated with the photocatalytic coating is illuminated with the wavelengths in the range between 340 nm and 450 nm at an intensity in the range of 1.0 W/m² to 20 W/m², or (iv) wherein a surface coated with the photocatalytic coating is illuminated with the wavelengths in the range between 340 nm and 450 nm at an intensity in the range of 1.0 W/m² to 10 W/m².

14-16. (canceled)

17. The enclosed space system of claim 1, wherein the light sources are or include one or more of fluorescent lamps, short wave lamps, gas discharge lamps, metal halide lamps and lasers.

18. The enclosed space system of claim 1, wherein the light sources are, or include, light emitting diodes (LEDs).

19-24. (canceled)

25. The enclosed space system of claim 1, (i) wherein the system is arranged to reduce an amount of NOx gases in the enclosed space, or (ii) wherein the system is arranged to reduce an amount of SOx gases in the enclosed space, or (iii) wherein the system is arranged to reduce an occurrence of bacteria and molds on the surface, or (iv) wherein the system is arranged to reduce an amount of volatile organic compounds gases in the enclosed space.

26-28. (canceled)

29. The enclosed space system of claim 1, wherein the photocatalytic coating is a cement-based hydraulic binding photocatalytic coating.

30. The enclosed space system of claim 1, wherein the photocatalytic coating is derived from a cement-based photocatalytic composition, which comprises:
(a) at least one cement binder;
(b) at least one photocatalyst;
(c) at least one cellulose ether;
(d) at least one fluidizing agent;
(e) at least one first calcareous filler in the form of particles of which at least 95% by weight has a dimension not greater than 100 nm;
(f) at least one second calcareous filler in the form of particles of which at least 95% by weight has a dimension not greater than 30 μm;
(g) at least one silane supported on an inorganic support in the form of powder.

31. The enclosed space system of claim 30, wherein the photocatalytic composition comprises:
(a) from 15 to 60% by weight, preferably from 20 to 50% by weight, of at least one cement binder;
(b) from 0.5 to 12% by weight, preferably from 1 to 8% by weight, of at least one photocatalyst;
(c) from 0.02 to 3% by weight, preferably from 0.05 to 1.5% by weight, of at least one cellulose ether;
(d) from 0.05 to 5% by weight, preferably from 0.1 to 2% by weight, of at least one fluidizing agent;
(e) from 10 to 50% by weight, preferably from 15 to 35% by weight, of at least one first calcareous filler in the form of particles of which at least 95% by weight has a dimension not greater than 100 μm;
(f) from 10 to 50% by weight, preferably from 15 to 35% by weight, of at least one second calcareous filler in the form of particles of which at least 95% by weight has a dimension not greater than 30 μm;
(g) from 0.05 to 5% by weight, preferably from 0.01 to 3% by weight, of at least one silane supported on an inorganic support in the form of powder.

32. The enclosed space system of claim 30, wherein the cement binder (a) is a Portland cement.

33. The enclosed space system of claim 30, wherein the photocatalyst (b) is photocatalytic titanium dioxide, mainly in anatase crystalline form.

34. The enclosed space system of claim 33, wherein the photocatalytic titanium dioxide has a granulometry such as at least 95% by weight has a dimension not higher than 50 nm, preferably not higher than 20 nm.

35. (canceled)

36. The enclosed space system of claim 30, wherein the cellulose ether (c) has a Brookfield viscosity RVT at 20° C. from 100 to 70,000 mPa˜s, preferably from 100 to 30,000 mPa˜s, more preferably from 200 to 10,000 mPa˜s.

37. The enclosed space system of claim 30, wherein the first calcareous filler (e) is in the form of particles of which at least 95% by weight has a dimension not greater than 70 μm, while the second calcareous filler (f) is in the form of particles of which at least 95% by weight has a dimension not greater than 20 μm.

38. The enclosed space system of claim 30, wherein the first calcareous filler (e) is in the form of particles of which not more than 5% by weight has a dimension not greater than 30 μm, preferably not greater than 20 μm.

39. The enclosed space system of claim 30, wherein the calcareous fillers (e) and (f) are present in a weight ratio (e)/(f) from 0.2 to 2.0, preferably from 0.5 to 1.5.

40. The enclosed space system of claim 30, wherein the supported silane (g) is in the form of particles of which at least 95% by weight has a dimension not greater than 100μ, preferably not greater than 80μ.

41. The enclosed space system of claim 30, further comprising: (h) at least one hydrophobized vinyl polymer, preferably a terpolymer of vinyichloride, ethylene and a vinyl ester CH₂═CH—O—C(═O)—R, wherein R is an alkyl, linear or branched, C₄-C₂₄.

42. The enclosed space system of claim 30, further comprising: (i) at least one salt of a long chain carboxylic acid.

43. The enclosed space system of claim 30, wherein water is added to the photocatalytic composition in a predetermined proportion, by mixing until a homogeneous and fluid product is obtained, and that product is applied to the interior surface of the enclosed space as the photocatalytic coating.

44. The enclosed space system of claim 43, wherein the weight ratio between water and cement binder (a) is from 0.2 to 0.8.

45. The enclosed space system of claim 30, wherein, after application and drying, the photocatalytic composition forms a coating layer having a thickness from 0.05 mm to 1 mm, preferably from 0.1 to 0.5 mm.

46. A method of using light emitting diodes emitting wavelengths in the range between 340 nm and 450 nm in a tunnel, the method including the step of: arranging in the tunnel the light emitting diodes emitting wavelengths in the range between 340 nm and 450 nm to illuminate an interior surface of the tunnel coated with a photocatalytic coating, wherein the photocatalytic coating is activatable by wavelengths in the range between 340 μm and 450 nm, and wherein the light emitting diodes do not emit light with wavelengths below 340 nm.

47. The method of claim 46, wherein the light emitting diodes emit wavelengths in the range between 340 nm and 389 nm.

48-53. (canceled)
